(19) United States
(12) Patent Application Publication
(10) Pub. No.: US 2004/0043083 A1
(43) Pub. Date: Mar. 4, 2004

(54) Anticancer composition comprising sesquiterpenes isolated from resina ferulae

(76) Inventors: Shi-Yong Ryu, Taejeon (KR); Chong-Ock Lee, Taejeon (KR); Sang-Un Choi, Taejeon (KR); Sung-Hee Park, Taejeon (KR); Young-Sup Kim, Taejeon (KR); Sung-Ki Kim, Taejeon (KR); Sang-Keun Kim, Kyungsangnam-do (KR); Shin-Kwon Kang, Kyungsangnam-do (KR)

Correspondence Address: David A Einhorn, Anderson Kill & Olick, 1251 Avenue of the Americas, New York, NY, 10020, US

(21) Appl. No.: 10/399,218

(22) PCT Filed: Oct. 10, 2001
(86) PCT No.: PCT/KR01/01697

(30) Foreign Application Priority Data
Oct. 12, 2000 (KR) 2000/60039

(51) Int. Cl. A61K 35/78

(52) U.S. Cl. 424/725

(57) ABSTRACT

A pharmaceutical composition comprising a lower alcohol-soluble extract of Ferula asafoedida L or a compound isolated therefrom including galbanic acid, karatavicinol, umbelliprenin, farnesiferol B, or farnesiferol C exhibits a potent anticancer activity.

### FIELD OF THE INVENTION

The present invention relates to an anticancer composition containing at least one sesguiterpene compound isolated from Resina Ferulae.

### BACKGROUND OF THE INVENTION

The practice of screening compounds isolated from the extracts of plants and microorganisms for their activities by way of conducting in vitro cell proliferation inhibition tests using cultivated cancer cell lines has seen some successes-in developing anticancer drugs such as taxol, vinblastin and camptothecin.

The term used in the Oriental medicine, “Regina Ferulae”, refers to dried resinous exudates obtained from the root and rhizoma of Ferula asafoedida L. and other plants belonging to the same genera in Umbelliferae which grow in China and Central Asia (Chung, B. S. et al., (1998) DOHAEHYANGYAKDAISAJEON (Plant part), Youngrim Press, pp422423). The exudates have been used to treat bronchochitis, asthma and pertussis (Ietsuo, A., (1985) CHfiuTGYAKDAISAJEON, Shanghai Science Technique Press, Vol. 1, p2); and galbanic acid or its sodium salt isolated from Ferula asafoedida L is known to inhibit the agglutination reaction of erythrocyte and platelet (Mansurov M. M. et al., (1990) Farmakol. Toksikol., 53, p51; Mansurov M. M. et al., (1988) Farmakol. Toksikol., S1, p47) and is also known to be effective in detoxifying the hepatotoxicity induced by injection of tetrachloromethane (Syrov V. N. et al., (1990) Farmakol. Toksikol., 53, p41).

The present inventors have endeavored to develop a new anticancer agent having a high potency, and have found that galbanic acid and other sesquiterpene compounds isolated from Regina Ferula show potent inhibiting activities against human cancer cell line proliferation

### SUMMARY OF THE INVENTION

Accordingly, it is an object of the present invention to provide a potent anticancer agent derived from Regina Ferula.

Another object of the present invention is to provide a pharmaceutical composition for preventing or treating cancer, comprising the anticancer agent.

A fierier object of the present invention is to provide a pharmaceutical composition for preventing or treating a cancer disease, comprising at least one member selected from the group consisting of galbanic acid, karatavicinol, umbelliprenin, farnesiferol B; farnesiferol C and pharmaceutically acceptable salts thereof, and a pharmaceutically acceptable carrier.

In accordance with one aspect of the present invention, there is provided a alcohol-soluble extract obtained by treating Regina Ferula with a lower alcohol.

In accordance with another aspect of the present invention, there are also provided galbanic acid, karatavicinol, umbelliprenin, farnesiferol B and farnesiferol C and pharmaceutically acceptable salts thereof isolated from the extract.

### DETAILED DESCRIPTION OF THE INVENTION

The alcohol-soluble extract may be prepared in a conventional manner from Ferula asafoedida L. or a plant of the same genera, such as Ferula conocaula Eug, Ferula narthex Boissier, Ferula foetida Regel, Ferula borealis X, Ferula fukanensis K. M. Shen, Ferula sinkiangensis K. M. Shen, and Ferula rigida W.

For example, Ferula Regina(resin) from Ferula asafoedida L. is sliced and dried in the shade. Then, an appropriate amount of a lower alcohol, e.g., methanol, ethanol or butanol, preferably 100% methanol, is added to the dried slices and the mixture is subjected to reflux-extraction for a period ranging from 30 min to 48 hrs, preferably from 1 br to 5 hrs, at a temperature ranging from 30 to 100° C., preferably 40 to 80° C. The extract is filtered and concentrated under a reduced pressure to obtain the alcohol-soluble extract.

Sesquiterpenes such as galbanic acid, karatavicinol, umbelliprenin, farnesiferol B and farnesiferol C may be isolated from the alcohol-soluble extract By way of conducting solvent fractionation using an appropriate organic solvent such as chloroform, dichloromethane and ethylacetate, preferably, dichloromethane, active fractions, preferably, a dichloromethane soluble fraction can be obtainer The fractions exhibiting inhibitory activity against human cancer cell lines are then each subjected to repeated fractionation by silica gel column chromatography using an appropriate solvent mixture, preferably a dichloromethane-methanol mixture having a mix ratio in the range of 50:1 to 100:1(v/v), to obtain galbanic acid, karatavicinol, umbelliprenin, farnesiferol B and farnesiferol C. Their respective structures can, be represented by the formulae given in the order of (1), (2), (3), (4) and (5), below:

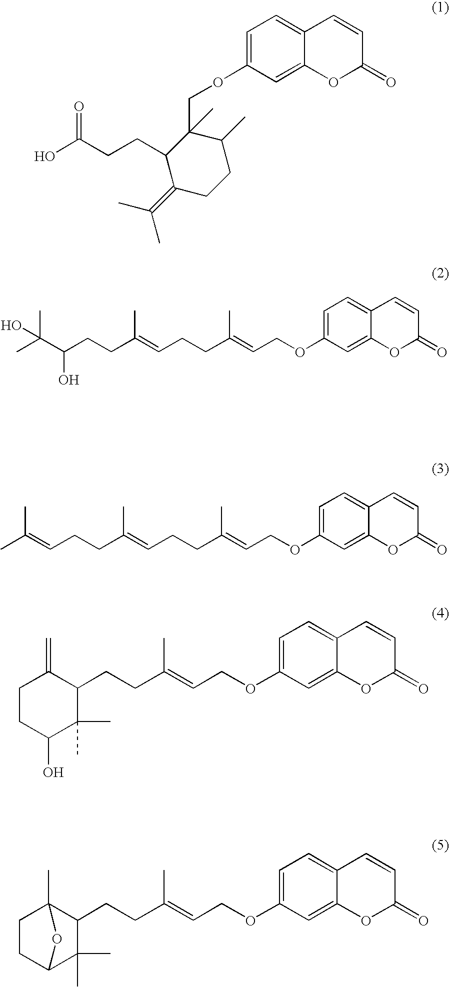

Each of the compounds has been found to inhibit human body derived cancer cell lines such as A549 (human lung cancer cell line), SK-OV-3 (human ovary cancer cell line), SK-MEL-2 (human melanoma cancer cell line), XF498 human CNS cancer cell line) and HCT-15 (human colic cancer cell line), and, therefore, they are expected to be effective for preventing and treating cancer diseases.

The pharmaceutically acceptable salt of each of the compounds may be a salt of an alkali metal, e.g., sodium and potassium, an alkali earth metal, e.g., magnesium and calcium, or ammonia or an organic base, e.g., TEA, pyridine and picoline.

The inventive pharmaceutical formulation may be prepared in accordance with one of the conventional procedures. In preparing the formulation, the active ingredient is preferably admixed or diluted with a carrier, or enclosed within a carrier which may be in the form of a capsule, sachet or other container. When the carrier serves as a diluent, it may be a solid, semi-solid or liquid material acting as a vehicle, excipient or medium for the active ingredient Thus, the formulation may be in the form of a tablet, pill, powder, sachet, elixir, suspension, emulsion, solution, syrup, aerosol, soft and hard gelatin capsule, sterile injectable solution, sterile packaged powder and the like.

Examples of suitable carriers, excipients, or diluents are lactose, dextrose, sucrose, sorbitol, mannitol, starches, gum acacia, alginates, gelatin, calcium phosphate, calcium silicate, cellulose, methylcellulose, microcrystalline cellulose, polyvinylpyrrolidone, water, methylhydroxybenzoates, propylhydroxybenzoates, talc, magnesium stearate and mineral oil. The formulation may additionally include fillers, anti-agglutinating agents, lubricating agents, wetting agents, flavoring agents, emulsifiers, preservatives and the like. The composition of the invention may be formulated so as to provide a quick, sustained or delayed release of the active ingredient after it is administrated to a patient, by employing any one of the procedures well known in the art.

The pharmaceutical formulation of the present invention can be administered via various routes including oral, transdermal, subcutaneous, intravenous and intramuscular introduction. For treating a human patient, a typical daily dose of the above-mentioned compounds isolated from Regina ferula may range from about 0.001 to 1 g/kg body weight, preferably 0.01 to 0.1 g/kg body weight, and can be administered in a single dose or in divided doses. However, it should be understood that the amount of the active ingredient actually administered ought to be determined in light of various relevant factors including the condition to be treated, the chosen route of administration, the age, sex and body weight of the individual patient, and the severity of the patient's symptom; and, therefore, the above dose should not be intended to limit the scope of the invention in any way.

The following Reference Example, Example and Test Example are intended to further illustrate the present invention without limiting its scope.

### Reference Example

### Inhibition Test Against the Proliferation of Human Cancer Cell Line

A549 (human lung cancer cell line), SK-OV-3 (human ovary cancer cell line), SK-MEL-2 (Han melanoma cancer cell line), XF498 (Human CNS cancer cell line) and HCT-15 (human colic cancer cell line) were obtained from NCI of U.S. A. and subjected to subculture. The inhibition of the cancer cell line proliferation was determined by the SRB (sulforhodamine B) method in accordance with the procedure described by Skehan, P. et al. (J. Nat'l. Canc. Inst., 82, p1107, 1990) as follows.

1) Each of the subcultured cancer cell lines was collected from the surface of the culture vessel, treated with a trypsin-CDTA solution, diluted, and added to each wall of a 96-well plat bottom microplate (Falcon Co.) in an amount of 5×10³ (A549, HCT-15), 1×10⁴ (SK-MEL-2, XF498) or 2×10⁴ (SK-OV-3) cells/well, followed by incubation in a CO₂ incubator to anchor the cells to the bottom surface. Then the culture medium was removed from the plate with suction, a solution containing a predetermined amount of a test sample (test solution) dissolved in culture medium was added to each well; and the mixture was futher cultured for 48 hours.

2) Thereafter, the culture medium was removed from the plate with suction, and 10 μl of 10% TCA (trichloro acetic acid) was added to each well. The plate was allowed to stand at room temperature for one hour to fix the cells, and was washed several times with distilled water to completely remove excess TCA and then dried.

3) Each well was treated with 100 μl of an SRB staining solution (0.4% suforhodamine in 1% acetic acid) for 30 minutes, washed several times with 1% acetic acid to remove the remaining staining solution, and dried at room temperature.

4) After adding 100 μl of 10 mM Trisma base solution (unbuffered) to each well, the plate was shaken with a titer plate shaker for 10 minutes to elute the staining solution from the cells, and the absorbance thereof at 520 nm was determined with a microplate reader.

5) The degree of cancer cell proliferation (% cell growth, i.e., reverse of anticancer activity) of the cell line treated with the test solution was calculated by the following numerical formula wherein C is the number of cells in a control group treated with a culture medium instead of the test solution; Tz, the number of cells at time zero; and T, the number of cells treated with the test solution calculated based on measurement. Numerical formula 1 was used when Tz>T, numerical formula 2, when Tz<T.

Numerical formula 1=[(T−Tz)/(C−Tz)]×100

Numerical formula 2=[(T−Tz)/Tz]×100

6) The amount of the samples needed for achieving 50% inhibition of the inhibiting cancer cell proliferation (ED₅₀) was calculated from the observed degree of cell proliferation at various concentrations of the sample using the data regression of LOTUS program.

### EXAMPLE

### Isolation of Compounds 1 to 5 from Ferula asafoedida L

Dried Regina Perulae (1.2 kg) of Ferula asafoedida L was subjected to 3 cycles. of reflux-extraction each with 20 l of methanol for 3 hrs, and then cooled. The extract was filtered and concentrated under a reduced pressure, i.e., to obtain a methanol soluble crude exbact (460 g).

The crude extract was dispersed in an appropriate amount of distilled water and subjected to solvent fractionation, successively with n-hexane, dichloromethane and n-butanol, to obtain an n-hexane soluble fraction (56 g), a dichloromethane soluble fraction (290 g) and an n-butanol soluble fraction (50 g). Five (5) gram of the dichloromethane soluble fraction was re-dissolved in 100 ml of dichloromethane and subjected to silica gel column chromatography (Merck, Kiesel gel 60, 70-230 mesh, 300 g, 3.0×80 cm) using successively dichloromethaie, a dichloromethane-methanol mixture, and methanol as tile eluent, to obtain four fractions: fraction 1 (480 mg) eluted with 2 l of dichloromethane; fraction 2 (2,050 mg) eluted with 2 l of a 100:1 mixture of dichloromethane and methanol; fraction 3 (570 mg) eluted with 2 l of a 50:1 mixture of dichloromethane and methanol; fraction 4 (980 mg) eluted with V of a 10:1 mixture of dichloromethane and methanol. The activity of each of the above fractions in inhibiting the proliferation of human cancer cell lines was measured by the procedure described in Reference Example. Fractions 2 and 3 were highly active, showing ED₅₀ values of about 8 μg/ml and 9 μg/ml, respectively.

In order to obtain the most active component, fractions 2 and fraction 3 were each subjected to repeated cycles of column chromatographic fractionation using n-hexane-ethyl acetate nixtures of varying ratios, finally to obtain two compounds isolated from fraction 2: 70 mg of umbelliprenin (3) in the form of colorless needle crystals (mp 61° C., C₂₄H₃₀O₃, M. W. 366); and 120 mg of farnesiferol B (4) in the form of colorless needle crystals {mp 112° C., [α]_{D}+10(c, 1.0 in CHCl₃), C₂₄H₃₀O₄, M.W. 382}, as well as three compounds isolated from fraction 3: 500 mg of galbanic acid (1) in the form of colorless needle crystals, {mp 87˜89° C., [α]_{D}-20(c, 1.0 in CHCl₃), C₂₄H₃₀O₅, M.W. 398}; 50 mg of karatavicinol (2) in the form of colorless crystals {mp 50° C., [α]_{D}-4(c, 1.0 in CHCl₃), C₂₄H₃₂O₅, M.W. 400}; and 400 mg of farnesiferol C (5) in the form of colorless needle crystals {mp 82° C., [α]_{D}-36(c, 1.0 in CHCl₃), C₂₄H₃₀O₄, M.W. 400}.

### Test Example

### Activities of Compounds 1 to 5 in Inhibiting the Proliferation of Human Cancer Cell Lines

The Inhibitory activities of the methanol extract and compounds 1 to 5 isolated from Ferula asafoedida L against the proliferation of each of five human cancer cell lines including A549 cell line were determined by the procedure described in Reference Example. The result is shown in Table 1, wherein cisplastine was used as the control drug.

TABLE 1
Inhibitory activities of the methanol extract of Ferula asafoedida L and
compounds 1 to 5 isolated therefrom against the proliferation of
cancer cells.

| | ED₅₀(μg/ml) | | | | |
|---|---|---|---|---|---|
| | A549 | SK-OV-3 | SK-MEL-2 | XF498 | HCT-15 |
| Methanol Extract | 11.5 | 18.6 | 13.0 | 11.2 | 12.0 |
| Compound 1 | 8.9 | 14.5 | 12.3 | 13.0 | 9.7 |
| Compound 2 | 5.6 | 11.6 | 5.7 | 7.5 | 7.0 |
| Compound 3 | 8.9 | 9.4 | 6.7 | 5.3 | 4.1 |
| Compound 4 | 7.2 | 10.0 | 7.1 | 7.1 | 4.3 |
| Compound 5 | 4.2 | 7.2 | 3.9 | 5.4 | 4.3 |
| Cisplastine | 1.8 | 1.5 | 1.2 | 1.4 | 3.2 |

As can be seen from Table 1, compounds 1 to 5 showed potent inhibitory activities against various cancer cell lines, among which compound 5 was the most

Cisplastine is known to have the problem of severe toxicity such as hepatotoxocity and renal-toxicity. On the other hand, Regina Ferulae has long been used as herbal medicine having no toxicity, and therefore, the inventive compounds isolated from Regina Ferala are expected to entail no adverse side effects.

While the invention has been described with respect to the above specific embodiments, it should be recognized that various modifications and changes may be made to the invention by those skilled in the art which also fall within the scope of the invention as defined in the appended claims.

What is claimed is:

1. An alcohol-soluble extract of Regina Ferulae having an anticancer activity.

2. The alcohol-soluble extract of claim 1, wherein said Regina Ferulae originates from a member selected from the group consisting of Ferula asafoedida L, Ferula conocaula Eug, Ferula narthex Boissier, Ferula foetida Regel, Ferula borealis K, Ferula fukanensis K. M. Shen, Ferula sinkiangensis K. M. Shen, and Ferula rigida W.

3. The alcohol-soluble extract of claim 1, wherein said Regina Ferula originates from Ferula asafoedida L.

4. The alcohol-soluble extract of claim 1, wherein the alcohol is selected from the group consisting of methanol, ethanol and butanol.

5. The alcohol-soluble extract of claim 3, wherein the alcohol is methanol.

6. A pharmaceutical composition for preventing or treating cancer, comprising the alcohol-soluble extract of claim 1, 2 or 3 and a pharmaceutically acceptable carrier.

7. The pharmaceutical composition of claim 6, wherein the alcohol is methanol.

8. A pharmaceutical composition for preventing or treating cancer, comprising at least one active ingredient selected from the group consisting of galbanic acid, karatavicinol, umbelliprenin, farnesiferol B, farnesiferol C and a pharmaceutically acceptable salt thereof, and a pharmaceutically acceptable carrier.

9. The pharmaceutical composition of claim 6, 7 or 8, wherein the cancer is lung cancer, ovary cancer, melanoma cancer or colic cancer.
